(19) United States
(12) Patent Application Publication
(10) Pub. No.: US 2017/0354438 A1
(43) Pub. Date: Dec. 14, 2017

(54) Systems and Methods of Callus Removal

(71) Applicant: Alfalfa Nail Supply, Inc.

(72) Inventor: Kevin Nguyen, Baton Rouge, LA (US)

(73) Assignee: Alfalfa Nail Supply, Inc., Baton Rouge, LA (US)

(21) Appl. No.: 15/181,368

(22) Filed: Jun. 13, 2016

(51) Int. Cl. A61B 17/54

(52) U.S. Cl. CPC A61B 17/54

(57) ABSTRACT

Example embodiments of the systems and methods of callus removal disclosed herein comprise a callus catching function to catch the calluses removed by the blade. A clamp on the back side of the blade comprises a hollowed out section into which the blade deposits the removed calluses. The hollowed area may be formed, for example, by a clamp for holding the blade in a handle. In an example embodiment, the clamp may also comprise a pumice sponge for further use in pedicure processes. The clamp may be removable to allow for removal of the collected calluses into a collection bin.

### TECHNICAL FIELD

The present disclosure is generally related to pedicures and, more particularly, is related to callus removal.

### BACKGROUND

A callus is a toughened area of skin which has become relatively thick and hard in response to repeated friction, pressure, or other irritation. Rubbing that is too frequent or forceful may cause blisters rather than allow calluses to form. Calluses are most often found on feet because of frequent walking. Calluses are generally not harmful, but may sometimes lead to other problems, such as skin ulceration or infection.

On the feet, calluses may form on the small toes due to the compression applied by tightly fitting shoes. Activities that are notorious for causing calluses include (but are not limited to) construction work, many sports, wood carving, playing musical instruments, use of a chef's knife, Trikke carving, rock climbing, hiking, martial arts, weight training, BMXing, dancing (especially ballet), chopping wood, and wearing high heels. Although often found on the foot (where the most pressure and friction are applied), calluses can occur anywhere on the body as a reaction to moderate, constant “grinding” pressure. It is the natural reaction of the palmar or plantar skin. Too much friction occurring too fast for the skin to develop a protective callus will cause a blister or abrasion instead.

Biologically, calluses are formed by the accumulation of terminally undifferentiated keratinocytes in the outermost layer of skin. Though the cells of calluses are dead, they are quite resistant to mechanical and chemical insults due to extensive networks of cross-linked proteins and hydrophobic keratin intermediate filaments containing many disulfide bonds.

Sometimes a callus occurs where there is no rubbing or pressure. These hyperkeratoses can have a variety of causes. Some toxic materials, such as arsenic, can cause thick palms and soles. Some diseases, such as syphilis, can cause thickening of the palms and soles as well as pinpoint hyperkeratoses. There is a benign condition called keratosis palmaris et plantaris, which produces corns in the creases of the fingers and non-weight bearing spaces of the feet. Some of this may be caused by actinic keratosis, which occurs due to overexposure to sun or with age and hormonal shifts.

Calluses and corns may go away by themselves eventually, once the irritation is consistently avoided. They may also be sanded down with a pumice stone or filed down with a callus shaver. Previous callus removal tools have proven inadequate at safely and hygienically removing calluses. There are heretofore unaddressed needs with these previous devices.

### SUMMARY

Example embodiments of the present disclosure provide systems of callus removal. Briefly described, in architecture, one example embodiment of the system, among others, can be implemented as follows: a callus catcher; and a housing configured to removably receive the callus catcher, the housing further configured to hold a callus removal blade, the blade comprising a front side configured to remove calluses and a back side configured to deposit the removed calluses in the callus catcher.

Embodiments of the present disclosure can also be viewed as providing methods for callus catcher. In this regard, one embodiment of such a method, among others, can be broadly summarized by the following steps: placing a callus removal blade in a callus remover housing; placing a clamp in a back side of the housing for mating against the blade, the clamp forming a hollowed area under the blade for collection of removed calluses.

### BRIEF DESCRIPTION OF THE DRAWINGS

FIG. 1 provides a top side view of an example embodiment of a system of callus removal.

FIG. 2 provides a bottom side view of an example embodiment of the system of FIG. 1.

FIG. 3 provides a top side view of an example embodiment of the system of FIG. 1.

FIG. 4 provides an exploded view of an example embodiment of the system of FIG. 1.

FIG. 5 provides an assembled view of an example embodiment of the disclosed systems of callus removal.

FIG. 6 provides a flow diagram of an example embodiment of a method of callus removal.

FIG. 7 provides a back side view of an example embodiment of the system of FIG. 1.

FIG. 8 is an exploded side view of an example embodiment of the clamp of FIG. 1.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

Example embodiments disclosed herein are largely described in terms of the performance of a pedicure, but are not intended to be limited to pedicures. Pedicures are used in the examples just for the purposes of illustration. When providing a pedicure, a scraping of the bottom of the foot, especially the heel, is often performed to remove calluses. A perforated blade section is moved across the heel to remove the calluses. As the blade removes the calluses, the dead skin is discarded, often to the floor, which may cause an unhygienic environment.

Example embodiments of the systems and methods of callus removal disclosed herein comprise a callus catching function to catch the calluses removed by the blade. A clamp on the back side of the blade, comprises a hollowed out section into which the blade deposits the removed calluses. The hollowed area may be formed, for example, by a clamp for holding the blade in a handle. In an example embodiment, the clamp may also comprise a pumice sponge for further use in pedicure processes. The clamp may be removable to allow for removal of the collected calluses into a collection bin.

In an example embodiment, the disclosed systems and methods of callus removal comprise a handle, a callus removal blade, and clamp. The handle may be of many different sizes and shapes, but is configured to removably receive the callus removal blade. The clamp may be configured to hold the blade in face as well as collect the calluses removed by the blade. In an example embodiment, the clamp comprises a pumice sponge.

In an example embodiment, tabs hold the clamp in the handle and the clamp comprises a hollow chamber to receive the shaved calluses, A tab on either end of the clamp may be used to enable the removal of the clamp from the handle.

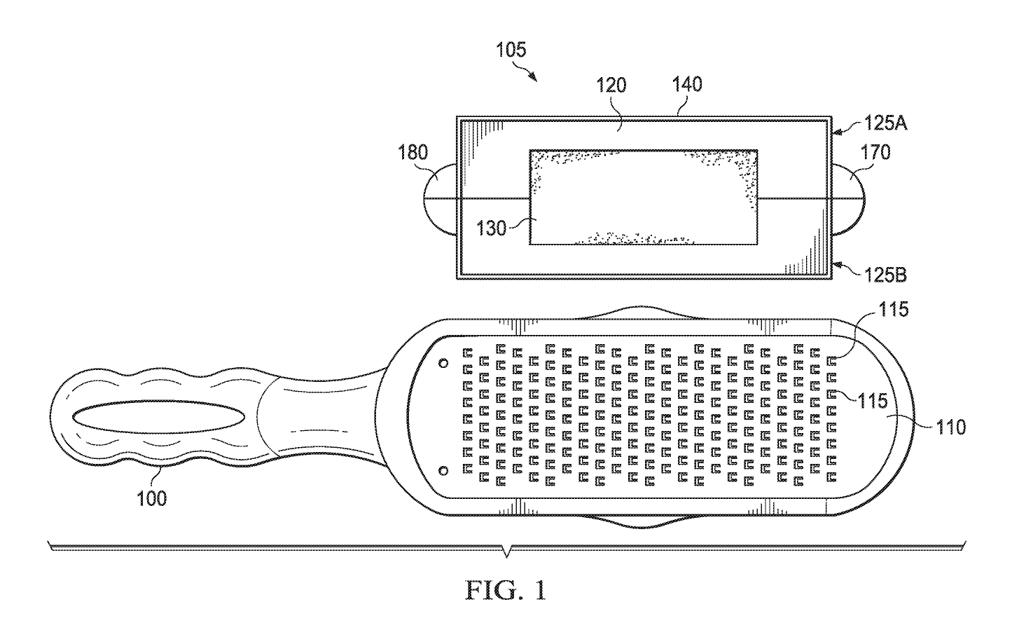

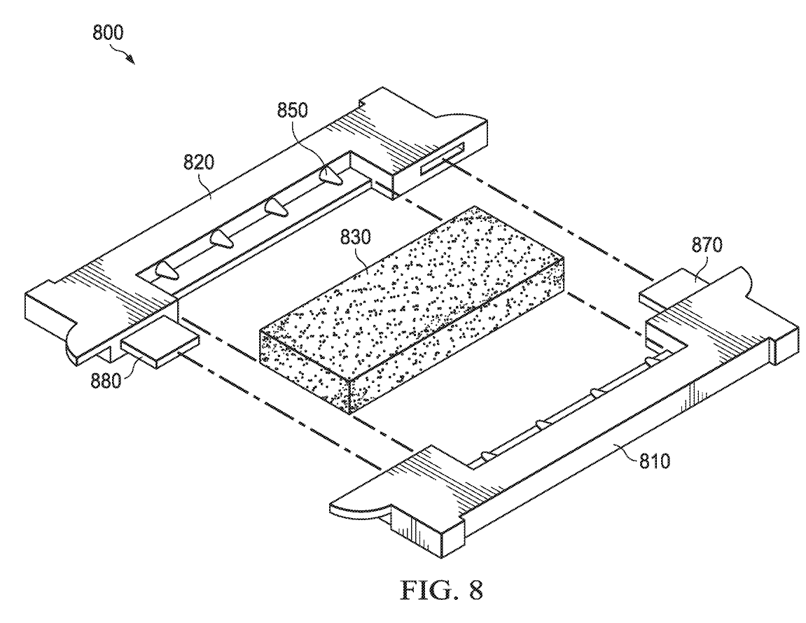

FIG. 1 provides a top-side view of handle 100 with callus removal blade structure 110 (such as a blade) removably installed. Callus removal blade structure is shown with squared off openings, but any shaped opening will work with the embodiments disclosed herein. Clamp 105 is configured to fit in the bottom side of handle 100 with receiving area 120 for collecting removed calluses. Clamp 105, including two “U” shaped pieces 125A, 125B, clamps implement 130, such as a pumice stone. Clamp 105 is further described below in connection to FIG. 8.

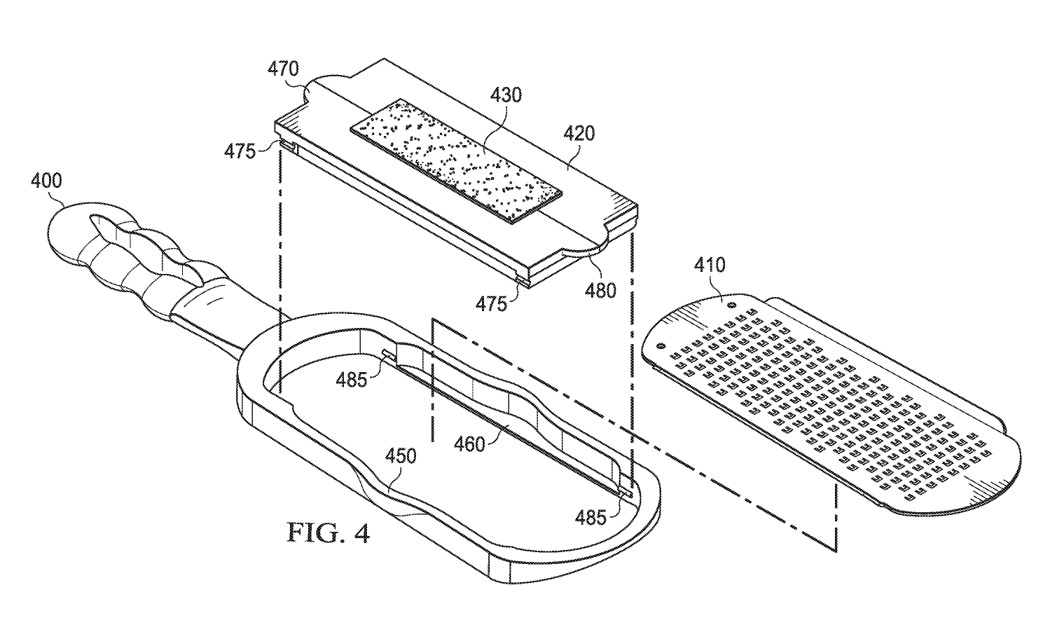

Clamp 105 includes raised structure 140 along the peripheral of clamp 105. Raised structure 140 abuts a back side of callus removal blade structure 110, an example embodiment of which is shown in FIG. 4. Raised structure 140 surrounds individual blades 115 such that it captured calluses that are removed by callus removal blade structure 110. Callus removal blade structure 110 may be constructed of stainless steel or other appropriate materials.

Tabs 170 and 180 are positioned on opposite ends of clamp 105. Tabs 170 and 180 may simultaneously provide stability and support for clamp 105 while also providing means for removal of clamp 120 from the bottom side of handle 100. Although, the example embodiments are shown with a long handle with which to hold the implement, no “handle” is actually necessary. Blade 110 and clamp 120 may be housed in a non-handled housing as well.

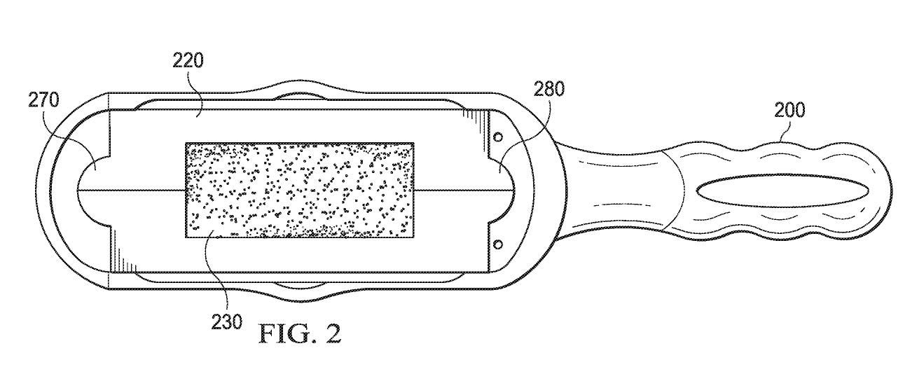

FIG. 2 provides a bottom side view of an example embodiment of the disclosed systems of callus removal. Clamp 220 is removably inserted into handle 200. With clamp 220 installed, an inner cavity is created by way of the back side of callus removal blade structure 110, receiving area 120, implement 130, and raised structure 140 (FIG. 1) for the collection of calluses removed by the callus removal blade (not shown). Tabs 270 and 280 provide stability for clamp 220 in handle 200. Tabs 270 and 280 also allow a means to pry clamp 220 out of handle 200. Removing clamp 220 from handle 200 allows for the removal of the collected calluses. Pumice stone 230 or other implement may be positioned in clamp 220 for performing other useful functions.

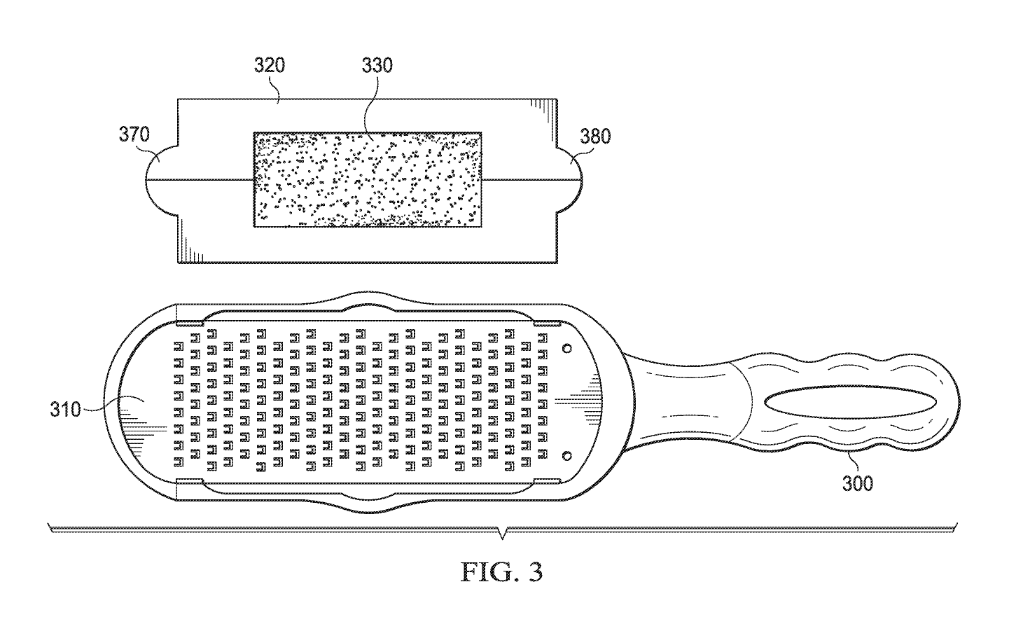

FIG. 3 provides a top side view of an example embodiment of a system of callus removal as disclosed herein. Handle 300 holds removable blade structure 310. Removal blade structure 310 may be a grater type of blade such that when removal blade structure 310 is rubbed across a surface, such as the bottom surface of a foot, any calluses or other dried skin areas are removed from the surface and pass through the blade. The removed calluses and skin particles may be collected in a receiving area of clamp 320, which fits on the back side of handle 300. Implement 330, shown as in FIG. 2, may comprise a pumice stone or other implement that is useful in pedicure services. Tabs 370 and 380 may serve to stabilize clamp 320 in handle 300. Tabs 370 and 380 may also be used to ease in the removal of clamp 320 from handle 300.

FIG. 4 provides an exploded view of an example embodiment of the disclosed systems of callus removal comprising handle 400, blade structure 410, and clamp 420. Blade structure 410 is removably placed into handle 400 and clamp 420 holds blade structure 410 in place. In an example embodiment, clamp 420 holds blade structure 410 against side rails 450 and 460. Alternatively or additionally, blade structure 410 may be attached to handle 400 without clamp 420 as rails 450 and 460 may hold blade structure 410 in position. Similarly, clamp 420 may be attached to handle 400 without blade structure 410 as rails 450 and 460 may hold clamp 420 in position. Alternatively or additionally, clamp 420 may be held in place in handle 400 with one or more protruding nibs 485 that fit into one or more corresponding indentations 475. Nibs 485 and corresponding indentations 475 may be any suitable shape or size. Also, whereas the example shown in FIG. 4 comprises nibs 485 on handle 400 and indentations 475 on clamp 420, the nibs may, instead, be on clamp 420 and indentations 475 may be on handle 400. In yet another embodiment, handle 400 may comprise both nibs and indentations and clamp 420 may have corresponding indentations and nibs. Implement 430, shown as in FIG. 2, may comprise a pumice stone or other implement that is useful in pedicure services. Tabs 470 and 480 may serve to stabilize clamp 420 in handle 400. Tabs 470 and 480 may also be used to ease in the removal of clamp 420 from handle 400.

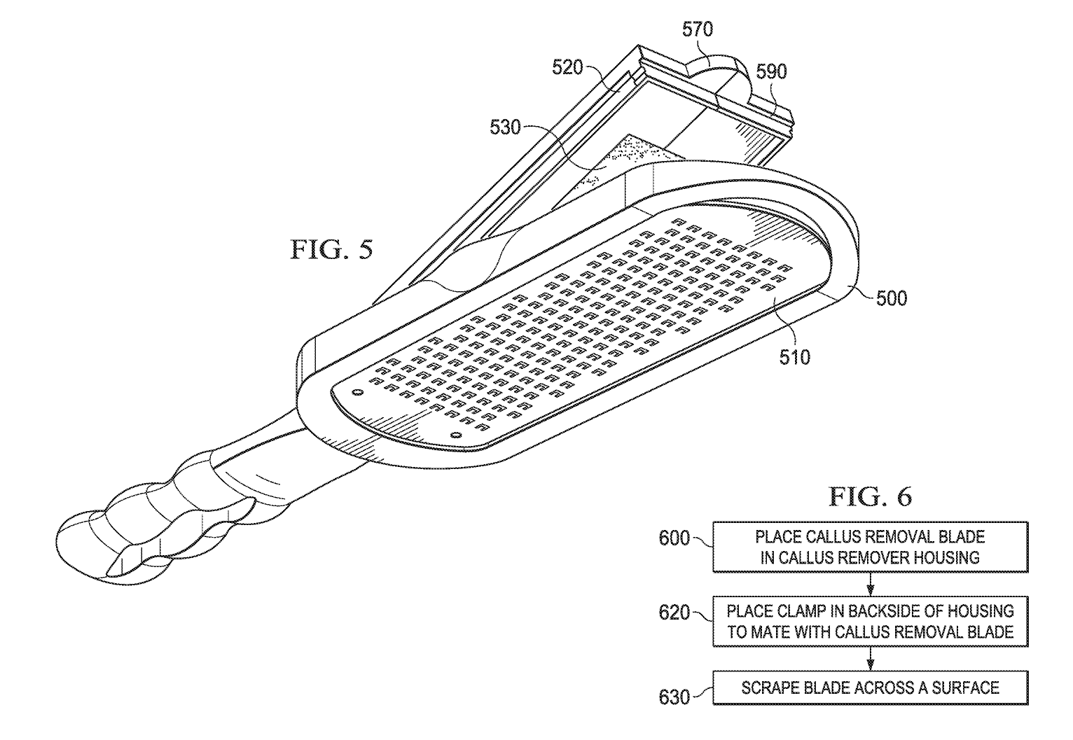

FIG. 5 provides an assembled view of an example embodiment of the disclosed systems of callus removal comprising handle 500, blade 510, and clamp 520. Clamp 520 comprises tab 570, edge 590 and pumice stone 530. When clamp 520 is placed in handle 500, the area under pumice stone 530 forms a hollowed area in which removed calluses from blade 510 are collected. Raised structure 590 abuts against blade structure 510 to hold blade structure 510 in handle 500.

FIG. 6 provides a flow diagram of a method of callus removal. In block 600, a callus removal blade is placed in the callus remover housing. In block 620, a clamp is placed in the back side of the housing and mates up against the blade. The clamp forms a hollowed area under the blade for collection of removed calluses. In block 630, the blade is scraped across a surface.

The flow chart of FIG. 6 shows the architecture, functionality, and operation of a possible implementation of a callus removal tool. It should also be noted that in some alternative implementations, the functions noted in the blocks may occur out of the order noted in FIG. 6. For example, two blocks shown in succession in FIG. 6 may in fact be executed substantially concurrently or the blocks may sometimes be executed in the reverse order, depending upon the functionality involved.

Back to the structure of the callus removal system, lateral sides 715 of blade 710 fit along ledges 760 along the inside lengths of handle 700. This allows blade 710 to fit snugly in handle 700 without falling out when used for scraping. Blade 710 may be manually removed with sufficient effort and without damaging blade 710 or handle 700. Indentation 750 may be located on the inside of one or both lateral sides of handle 700 to aid in removal of blade 710. The clamp may then be inserted into the handle, snapped into place, covering the back side of the blade 710. Clamp may be held in place by one or more protruding nibs such that the clamp does not freely disengage from handle 700. Alternatively, the clamp may comprise one or more nibs on each lateral side that fit into corresponding indentations in handle 700. However, the clamp may be manually removed with sufficient effort and without damaging the clamp or handle 700.

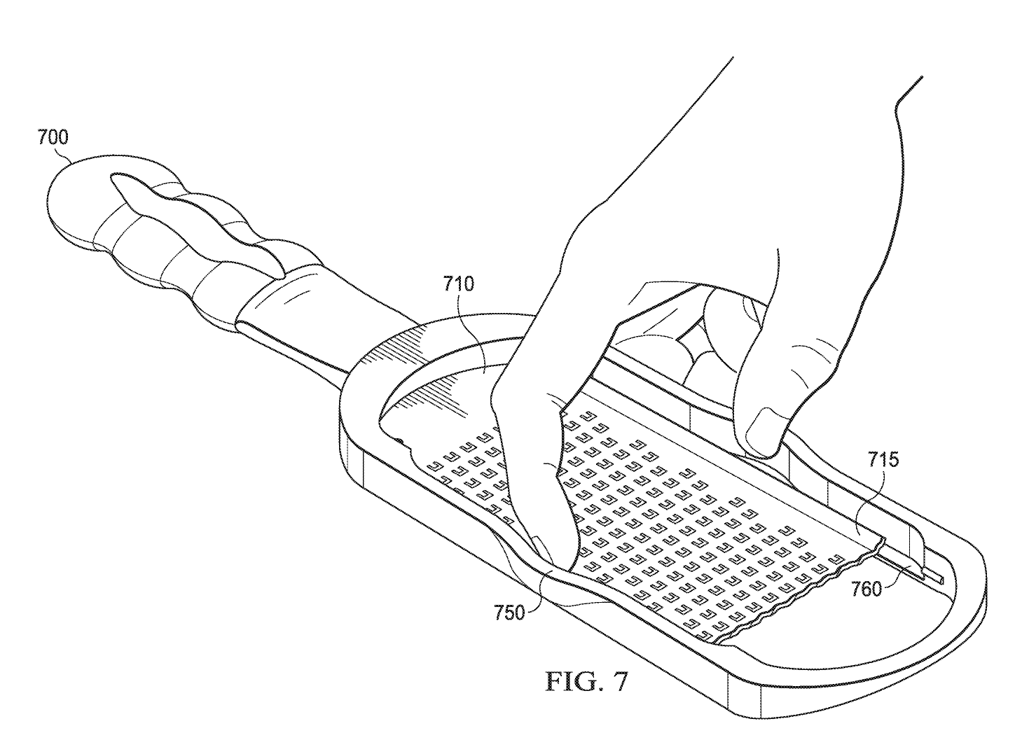

FIG. 8 provides an exploded view of an example embodiment of the clamp of FIG. 1. In an example embodiment, clamp 800 comprises two pieces 810, 820 (such as two halves), such that implement 830, such as a pumice stone, may be inserted into first piece 810, and second piece 820 may then be snapped together with first piece 810 to hold implement 830. Each half 820, 830 may comprise a tab 870, 880, that fits in an opening in the opposite piece to secure the two halves together. In an example embodiment, each piece 810, 820 comprises means 850 for securing implement 830 between pieces 810, 820. Means 850 may comprise any type of abutment to penetrate implement 830 or otherwise secure implement 830 in clamp 800. Means 850 may be situated along the lateral sides or in any location that would assist in securing implement 830. Clamp 800, including pieces 810, 820, and implement 830, acts as a callus catcher for the skin removed through blade 710 of FIG. 7.

Although the present disclosure has been described in detail, it should be understood that various changes, substitutions and alterations can be made thereto without departing from the spirit and scope of the disclosure as defined by the appended claims.

Therefore, at least the following is claimed:

1. A system comprising:
a callus catcher; and
a housing configured to removably receive the callus catcher, the housing further configured to hold a callus removal blade, the blade comprising a front side configured to remove calluses and a back side configured to deposit the removed calluses in the callus catcher.

2. The system of claim 1, wherein the callus catcher comprises a pumice sponge.

3. The system of claim 2, wherein a front side of the pumice sponge is exposed for use of the sponge and a back side of the pumice sponge is exposed to the back side of the callus removal blade.

4. The system of claim 1, wherein the callus removal blade is removable from the housing.

5. The system of claim 1, wherein the callus catcher snaps into the housing forming an enclosed void between the housing and the callus catcher.

6. The system of claim 1, wherein the callus catcher comprises tabs on opposing ends, the tabs configured to stabilize the callus catcher in the housing.

7. The system of claim 1, wherein the housing comprises a handle

8. A system comprising:
means for removing calluses from a surface; and
removably attached means for collecting the removed calluses in an enclosed void.

9. The system of claim 8, wherein the means for removing calluses and the means for collecting the removed calluses are contained within a single implement.

10. The system of claim 8, wherein the means for collecting comprises a pumice stone.

11. The system of claim 8 further comprising a housing, the means for removing calluses and the means for collecting the removed calluses removably contained in the housing.

12. The system of claim 11, wherein the housing comprises a handle.

13. The system of claim 11, wherein the means for collecting comprises tabs on opposing ends, the tabs configured to stabilize the means for collecting in the housing.

14. The system of claim 8, wherein the means for removing and the means for collecting are positioned together to form a void into which the removed calluses are collected

15. A method comprising:
placing a callus removal blade in a callus remover housing;
placing a clamp in a back side of the housing for mating against the blade, the clamp forming a hollowed area under the blade for collection of removed calluses.

16. The method of claim 15, further comprising scraping the blade across a surface.

17. The method of claim 15, wherein the housing comprises a handle.

18. The method of claim 15, wherein the clamp comprises a pumice sponge.

19. The method of claim 15, further comprising stabilizing the clamp in the housing with tabs on opposing ends of the catcher.
